(19) United States
(12) Patent Application Publication
(10) Pub. No.: US 2021/0315737 A1
(43) Pub. Date: Oct. 14, 2021

(54) ANTERIOR CHAMBER MAINTAINER (ACM)

(71) Applicant: JEFFREY WHITSETT, Houston, TX (US)

(72) Inventor: JEFFREY WHITSETT, Houston, TX (US)

(21) Appl. No.: 17/220,858

(22) Filed: Apr. 1, 2021

Related U.S. Application Data
(60) Provisional application No. 63/008,022, filed on Apr. 10, 2020.

(51) Int. Cl. A61F 9/007; A61F 9/00

(52) U.S. Cl. CPC A61F 9/00781; A61F 9/0017

(57) ABSTRACT

An anterior chamber maintainer (ACM), which is to be utilized in connection with maintaining physiological intraocular pressure during intraocular surgery is disclosed and is seen to comprise an axially elongated tubular member which is adapted to be fluidically connected at a first end to a supply of balanced salt solution (BSS) for introduction into the anterior chamber of an eye so as to maintain adequate intraocular pressure while performing any intraocular surgery. A second opposite end of the anterior chamber maintainer (ACM) is provided with a sharply pointed end for insertion through a paracentesis formed within the limbus region of the eye, and a deformable bulbous portion is interposed between the first and second ends such that as the second end passes through the paracentesis, the bulbous portion will diametrically deform and then diametrically expand, after also passing through the paracentesis, thereby trapping the anterior chamber maintainer (ACM) within the anterior chamber of the eye.

### CROSS REFERENCE TO RELATED APPLICATIONS

This United States patent application is a non-provisional patent application based upon United States provisional application, Application Number 63/008,022, which was filed on Apr. 10, 2020, the priority benefits of which are hereby claimed.

### FIELD OF THE INVENTION

The present invention relates generally to medical equipment, and more particularly to a new and improved anterior chamber maintainer (ACM) which is to be utilized in connection with maintaining physiological intraocular pressure during intraocular surgery. Still more particularly, the new and improved anterior chamber maintainer (ACM) effectively comprises a device which enables the cataract surgeon to infuse balanced salt solution (BSS) into the anterior chamber of the eye so as to maintain adequate intraocular pressure while performing any intraocular surgery.

### BACKGROUND OF THE INVENTION

Conventionally, anterior chamber intraocular chamber maintainers (ACMs) have been used during different types of intraocular surgeries all of which require that the intraocular pressure be maintained within the anterior segment of the eye while performing such intraocular surgery. Such intraocular surgery may comprise, for example, cataract surgery wherein lens material is removed from the eye, or vitrectomy surgery wherein vitreous material such as, for example, floaters, are cut and removed from the posterior segment of the eye. Such conventional anterior chamber intraocular pressure maintainers (ACMs) utilize balanced salt solution (BSS) infused into anterior chamber of the eye. The constant infusion of balanced salt solution (BSS), and the maintenance of stable intraocular pressure within the eye, are both necessary in order to achieve proper fluidics and function during all intraocular surgeries. During cataract surgery, which is the most common ophthalmic surgical procedure performed within this country, balanced salt solution (BSS) is infused in a coaxial manner to inflate the eye while simultaneously removing the natural crystalline lens. During vitrectomy surgery, however, an infusion cannula is inserted through the pars plana of the eye so as to maintain the intraocular pressure while the vitreous material is being severed and removed. The problem with such an infusion cannula, however, resides in the fact that in order to render such fixed or stable relative to the eye, the infusion cannula is actually sutured to the pars plana. Therefore, not only is it necessary for the infusion cannula to be sutured to the sclera in the region of the pars plana, but then after the vitrectomy has been completed, the sutures will be required to close the scleral opening. All of these procedures not only require additional time to be invested by means of the vitrectomy surgeon and his or her staff, but such procedures significantly increase the morbidity rate as well as being more adversely impactful upon the patient's recovery.

A need therefore exists for a new and improved anterior chamber maintainer (ACM). Another need exists in the art for a new and improved anterior chamber maintainer (ACM) which can be used during different types of intraocular surgeries all of which require that the intraocular pressure be maintained within the anterior segment of the eye while performing such intraocular surgery. Still another need exists in the art for a new and improved anterior chamber maintainer (ACM) which can be used during different types of intraocular surgeries all of which require that the intraocular pressure be maintained within the anterior segment of the eye while performing such intraocular surgery and which is easy to manipulate by the surgeon. Yet another need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, and which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries.

A further need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye. A still further need exists in the art for a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye such that there are no sutures to be removed by the surgeon or any member of his or her staff which will lead to a more positive surgical result for the patient with respect to morbidity and recovery.

### Overall Objectives of the Present Invention

An overall objective of the present invention is to provide a new and improved anterior chamber maintainer (ACM). Another overall objective of the present invention is to provide a new and improved anterior chamber maintainer (ACM) which can be used during different types of intraocular surgeries all of which require that the intraocular pressure be maintained within the anterior chamber of the eye while performing such intraocular surgery. Still another overall objective of the present invention is to provide a new and improved anterior chamber maintainer (ACM) which can be used during different types of intraocular surgeries all of which require that the intraocular pressure be maintained within the anterior segment of the eye while performing such intraocular surgery and which is easy to manipulate by the surgeon. Yet another overall objective of the present invention is to provide a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, and which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries.

A further overall objective of the present invention is to provide a new and improved anterior chamber maintainer (ACM) which can be used during different types of intraocular surgeries all of which require that the intraocular pressure be maintained within the anterior segment of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye. A still further overall objective of the present invention is to provide a new and improved anterior chamber maintainer (ACM) which can be used during different types of intraocular surgeries all of which require that the intraocular pressure be maintained within the anterior chamber of the eye while performing such intraocular surgery, which is easy to manipulate by the surgeon, which comprises an infusion cannula to supply balanced salt solution (BSS) into the anterior chamber of the eye in view of the fact that the constant infusion of balanced salt solution (BSS), and the maintenance of stable intraocular pressure within the eye, are both necessary in order to achieve proper fluidics and function during all intraocular surgeries, and which is capable of effectively being fixed to or disposed in a stable state relative to the eye without the need for suturing the infusion cannula to any portion of the eye such that there are no sutures to be removed by the surgeon or any member of his or her staff which will lead to a more positive surgical result for the patient with respect to morbidity and recovery.

### SUMMARY OF THE INVENTION

The foregoing and other objectives are achieved in accordance with the principles and teachings of the present invention through means of an anterior chamber maintainer (ACM) which comprises a toggle or relatively large diameter tubular member which not only defines a fluid conduit for receiving an externally disposed sleeve member or infusion line, which may be fabricated from any conventional medical grade irrigation tubing and which is fixedly connected to a continuous supply of balanced salt solution (BSS) to be introduced into the anterior chamber of the eye, but in addition, the toggle or relatively large diameter tubular member effectively defines a handle which may be grasped by the surgeon to easily manipulate the anterior chamber maintainer (ACM). A one millimeter (1 mm) paracentesis is made at the limbus of the eye, and it is seen that the toggle or relatively large diameter tubular member is integrally mounted upon a base member or platform which is adapted to be engaged with an external portion of the eye when a distal end portion of a relatively small diameter tubular member, integrally connected to the base member or platform and provided with a sharply pointed end, is inserted through the paracentesis. In addition, an axially central portion of the relatively small diameter tubular member, that is located between the base member and the sharpened or pointed end, is provided with a deformable bulbous portion. The tubular member is fabricated from a suitable medical grade plastic material, such as, for example, 23 g plastic material, which is similar to that utilized to form IV angio-catheters and which exhibits sufficient axial stiffness to permit the tubular member to pass through the paracentesis and yet sufficient radial pliability and inherent resiliency and/or elastic memory to permit the bulbous portion to be deformed as it passes through the paracentesis and yet regain its original bulbous configuration once it has passed through the paracentesis.

More particularly, as the distal end of the relatively small diameter tubular member passes through the paracentesis, the bulbous portion will be forced to contract radially inwardly so as to permit the axially central portion of the tubular member to pass through the paracentesis, however, as soon as the bulbous member has effectively passed through the paracentesis, the bulbous member will regain its bulbous configuration. In this manner, it is further appreciated that as a result of the bulbous portion regaining its original configuration, the bulbous portion will now effectively stabilize the anterior chamber maintainer (ACM) within the anterior chamber of the eye such that the infusion line or sleeve member is now effectively connected to the eye whereby the balanced salt solution (BSS) can be fluidically conducted into the anterior chamber of the eye. This connection of the infusion line to the eye, by means of the new and improved anterior chamber maintainer (ACM) also permits the surgeon to grasp the anterior chamber maintainer (ACM) by means of the sleeve or infusion line and the relatively large diameter tubular member so as to effectively rotate, pivot, or otherwise directionally orient or manipulate the eye so as to aid the surgeon's visualization during performance of the surgery without any fear that the infusion line will become disconnected from the eye. When the surgery is complete, the anterior chamber maintainer (ACM) can be readily retracted out from the eye by simply pulling on the infusion line/anterior chamber maintainer (ACM) assembly whereby the axially central bulbous portion of the anterior chamber maintainer (ACM) will again radially deform so as to permit the anterior chamber maintainer (ACM) to pass back outwardly through the paracentesis.

### BRIEF DESCRIPTION OF THE DRAWINGS

Various other features and attendant advantages of the present invention will be more fully appreciated from the following detailed description when considered in connection with the accompanying drawing wherein:

FIG. 1 is a schematic cross-sectional view of a human eye schematically illustrating the insertion of the new and improved anterior chamber maintainer (ACM) when inserted into the eye; and

FIG. 2 is an enlarged, schematic front elevational view of the new and improved anterior chamber maintainer (ACM) clearly illustrating the structure of the anterior chamber maintainer (ACM) as comprising its toggle or relatively large diameter tubular member which comprises a plurality of serially arranged frusto-conical portions which define lower end annular stepped portions over which the infusion line is disposed and tightly secured, the sharply pointed distal end of the anterior chamber maintainer (ACM) which is adapted to be inserted through the paracentesis, and the bulbous portion of the anterior chamber maintainer (ACM) which passes through the paracentesis and maintains the anterior chamber maintainer (ACM) within the eye;

FIG. 3 is an enlarged, schematic cross-sectional view of the new and improved anterior chamber maintainer (ACM) as taken along the lines 3-3 of FIG. 2, clearly showing the sharply pointed distal end portion of the new and improved anterior chamber maintainer (ACM) which is adapted to easily pierce the paracentesis formed within the limbus portion of the eye;

FIG. 4 is an enlarged, schematic view of the encircled region 4 as shown in FIG. 3 so as to more clearly show the lower distal end portion of the new and improved anterior chamber maintainer (ACM) which includes the sharply pointed end portion and the bulbous portion of the new and improved anterior chamber maintainer (ACM).

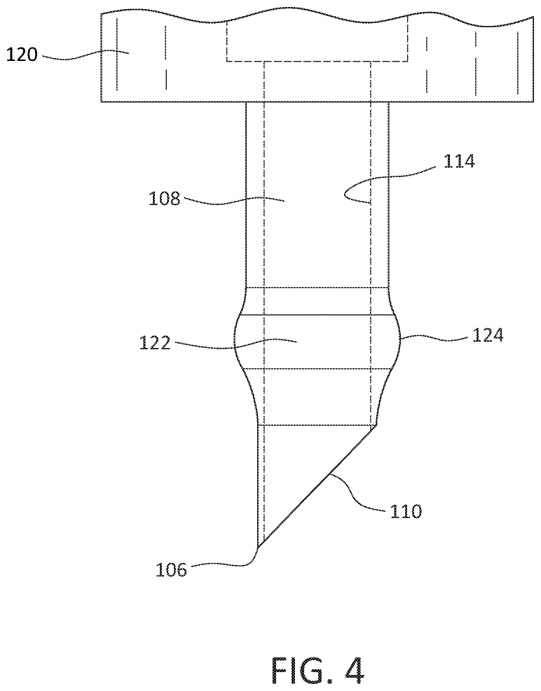

### DETAILED DESCRIPTION OF THE PRESENT INVENTION

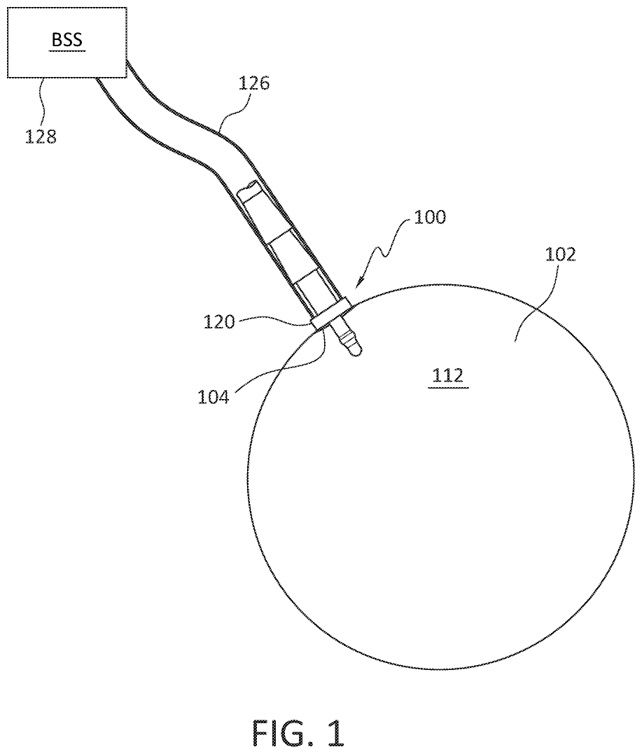

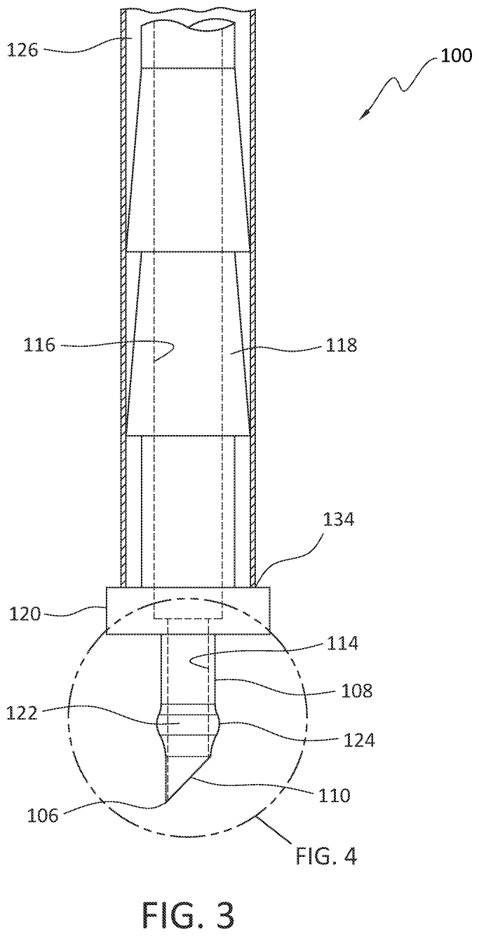

With reference being made to FIGS. 1,3 and 4, the new and improved anterior chamber maintainer (ACM) of the present invention is disclosed, is generally indicated by the reference character 100, and is schematically illustrated as it would be secured to and within a person's eye 102 during, for example, vitrectomy surgery. More particularly, a paracentesis 104 of approximately one millimeter (1 mm) is created at the limbus section of the eye 102, which is defined as the junction of the cornea and sclera of the eye, and a sharply pointed distal end portion 106 of a relatively small diameter tubular member 108 of the anterior chamber maintainer (ACM) 100, as can best be appreciated from FIG. 3, is adapted to be inserted through the paracentesis 104 such that an open end portion 110 of the sharply pointed distal end portion 106 of the anterior chamber maintainer (ACM) 100 is disposed within the anterior chamber 112 of the eyeball 102. Still further, it is seen that the relatively small diameter tubular member 108 defines an internal tubular fluid conduit 114 which is in fluidic communication with a larger, internal tubular fluid conduit 116 which is defined within a relatively large diameter tubular member 118. The relatively small diameter tubular member 108 and the relatively large diameter tubular member 118 are integrally connected together by means of a transversely oriented base member or platform 120 which, as can be appreciated from FIG. 1, is adapted to be seated upon an exterior surface portion of the eye 102 once the sharply pointed distal end portion 106 of the anterior chamber maintainer (ACM) 100 has been inserted through the paracentesis 104. It is also seen that an axially central portion 122 of the relatively small tubular member 108 comprises a deformable bulbous portion 124.

The relatively small diameter tubular member 108 is fabricated from a suitable medical grade plastic material, such as, for example, 23 g plastic material, which is similar to that utilized to form IV angio-catheters and which exhibits sufficient axial stiffness to permit the relatively small diameter tubular member 108 to pass through the paracentesis 104 and yet sufficient radial flexibility and inherent resiliency and/or elastic memory to permit the bulbous portion 124 to be deformed as it passes through the paracentesis 104 and yet regain its original bulbous configuration 124 once it has passed through the paracentesis 104. More particularly, as the relatively small diameter tubular member 106 passes through the paracentesis 104, the bulbous portion 124 will be forced to contract radially inwardly so as to permit the axially central portion 122 of the relatively small diameter tubular member 108 to pass through the paracentesis 104, however, as soon as the bulbous member or portion 124 has effectively passed through the paracentesis 104, the bulbous member or portion 124 will regain its bulbous configuration. In this manner, it is further appreciated that as a result of the bulbous portion 124 regaining its original configuration, the bulbous portion 124 will now effectively trap the anterior chamber maintainer (ACM) 100 within the anterior chamber 112 of the eye 102 such that an infusion line 126, as can best be seen in FIGS. 1 and 3, can be effectively connected to the relatively large diameter tubular member 118 of the anterior chamber maintainer (ACM) 100 whereby a balanced salt solution (BSS) can be fluidically conducted into the anterior chamber 112 of the eye 102 from a suitable source of balanced salt solution (BSS) 128.

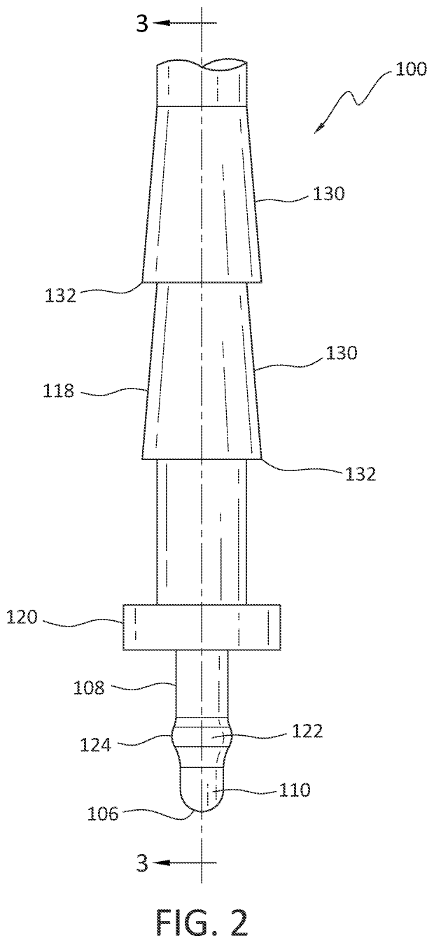

Continuing further, and with particular reference being made to FIGS. 2 and 3, it is seen that the relatively large diameter tubular section 118 of the anterior chamber maintainer (ACM) 100 comprises a plurality of frusto-conical portions or segments 130 integrally connected together and which define lower end annular stepped portions 132 over which the infusion line 126 is disposed and tightly secured in view of the fact that the infusion line 126 will conform to the external geometries of the frusto-conical portions or segments 130 whereby the annular stepped portions 132 will effectively prevent the infusion line 126 from becoming disconnected from the relatively large diameter tubular section 118 of the anterior chamber maintainer (ACM) 100. As a result of the aforenoted structure, the surgeon, performing a particular intraocular surgery, is able to easily grasp the anterior chamber maintainer (ACM) 100 by means of the relatively large diameter tubular member 118, and the infusion line 126 fluidically connected thereto, so as to effectively rotate, pivot, or otherwise directionally orient or manipulate the anterior chamber maintainer (ACM) 100 and the eye 102 in any particular direction and within any plane. These movements aid the visualization of the intraocular surgeon while he or she is performing the intraocular surgery without any fear that the infusion line 126 will become disconnected from the eye 102. It is lastly noted that the anterior chamber maintainer (ACM) 100 can be readily retracted out from the eye 102 by simply pulling on the infusion line/anterior chamber maintainer (ACM) assembly 126/100 whereby the axially central bulbous portion 124 of the anterior chamber maintainer (ACM) 100 will again be radially deformed so as to permit the anterior chamber maintainer (ACM) 100 to pass back outwardly through the paracentesis 104. It is lastly noted that when the infusion line 126 is mounted upon the relatively large diameter tubular section 118 of the anterior chamber maintainer (ACM) 100, the open end portion of the infusion line 126 will be seated upon the upper surface portions of the transversely oriented base member or platform 120 as at 134 shown in FIG. 3.

Obviously, many variations and modifications of the present invention are possible in light of the above teachings. It is therefore to be understood that within the scope of the appended claims, the present invention may be practiced otherwise than as specifically described herein.

What is claimed and desired to be protected by Letters Patent is:

1. A new and improved anterior chamber maintainer (ACM) for fixation within an eye during intraocular surgery, comprising:
an axially elongated tubular member comprising a first open end portion which is adapted to be fluidically connected to a balanced salt solution (BSS) infusion line for receiving a supply of balanced salt solution (BSS) to be introduced into an anterior chamber of an eye during intraocular surgery;
said axially elongated tubular member comprising a second open end portion, opposite said first end portion, adapted to be disposed within the anterior chamber of the eye such that the balanced salt solution (BSS) from said balanced salt solution (BSS) infusion line can enter the anterior chamber of the eye so as to provide and maintain adequate pressure within the anterior chamber of the eye during intraocular surgery; and
a bulbous portion which is mounted upon said axially elongated tubular member so as to be interposed between said first and second open end portions of said axially elongated tubular member and which is adapted to be radially compressed or contracted after said second open end portion of said axially elongated tubular member has passed through a paracentesis incision made within the eye and which will regain said bulbous configuration once said bulbous portion of said axially elongated tubular member has passed through the paracentesis such that said bulbous portion of said axially elongated tubular member effectively traps said anterior chamber maintainer (ACM) within the eye such that balanced salt solution (BSS) can be continuously supplied to the anterior chamber of the eye without fear that said balanced salt solution (BSS) infusion line will be fluidically disconnected from the anterior chamber of the eye.

2. The anterior chamber maintainer (ACM) as set forth in claim 1, wherein:
said second open end portion of said axially elongated tubular member comprises a sharply pointed distal end portion for facilitating insertion of said second open end portion and said bulbous portion of said axially elongated tubular member through the paracentesis formed within the eye.

3. The anterior chamber maintainer (ACM) as set forth in claim 1, wherein:
said first open end portion of said axially elongated tubular member is defined upon a first relatively large diameter tubular portion of said axially elongated tubular member; and
said second open end portion of said axially elongated tubular member is defined upon a second relatively small diameter tubular portion of said axially elongated tubular member.

4. The anterior chamber maintainer (ACM) as set forth in claim 3, wherein:
said first relatively large diameter tubular portion of said axially elongated tubular member comprises a plurality of frusto-conical portions or segments having annular stepped portions which engage said balanced salt solution (BSS) infusion line such that said balanced salt solution (BSS) infusion line is not easily separated from said first relatively large diameter tubular portion of said axially elongated tubular member such that fluid flow of said balanced salt solution (BSS) from said infusion line into the anterior chamber of the eye is ensured.

5. The anterior chamber maintainer (ACM) as set forth in claim 3, wherein:
said first relatively large diameter tubular portion of said axially elongated tubular member effectively defines a handle by means of which the surgeon may manipulate said anterior chamber maintainer (ACM) in any direction, within any plane, once said bulbous portion has passed through the paracentesis and is disposed within the anterior chamber of the eye.

6. The anterior chamber maintainer (ACM) as set forth in claim 1, wherein:
said axially elongated tubular member is fabricated from 23 g medical equipment tubular material.

7. The anterior chamber maintainer (ACM) as set forth in claim 1, wherein
said infusion line is fabricated from medical grade irrigation tubing.
